(19) United States
(12) Patent Application Publication
(10) Pub. No.: US 2007/0243155 A1
(43) Pub. Date: Oct. 18, 2007

(54) Skin care method and products

(76) Inventors: Pierre Bottiglieri, Aigle (CH); Robert E. Saute, Pacoima, CA (US); Candace R. Keefe, Irvine, CA (US); Antoaneta Abbruzzese, Irvine, CA (US)

Correspondence Address: KENNETH J. HOVET;NORDMAN, CORMANY, HAIR & COMPTON LLP, P.O. BOX 9100, 1000 TOWN CENTER DRIVE, OXNARD, CA, 93031-9100, US

(21) Appl. No.: 11/406,745

(22) Filed: Apr. 18, 2006

(51) Int. Cl. A61K 8/97; A61K 8/55

(52) U.S. Cl. 424/70.23; 424/74

(57) ABSTRACT

A skin treatment regimen comprising applying to facial skin, in order, at least once per day, effective amounts of five distinct products, including a daily cleanser, an exfoliating masque with thermal infusion, a daily balancer, a daily eye cream and a daily moisturizing cream.

### BACKGROUND OF THE INVENTION

1. Field of the Invention

The present invention pertains generally to skin care products. More particularly, the invention relates to skin care preparations and regimens for applying skin care products sequentially to maximize the therapeutic skin care effect.

2. Description of Related Art

The skin is a major bodily organ that performs a variety of functions, including the maintenance of moisture within the body. The skin also acts as a barrier to foreign objects, sunlight, chemical pollutants, and pathogens, all of which can have deleterious effects on the body. Skin on the face may be dry, oily, or a combination of oily and dry areas.

### SUMMARY OF THE INVENTION

The present invention overcomes many of the prior art omissions by providing a skin care regimen that self-adjusts to, and is effective on, oily skin, dry skin, and combination skin, i.e., skin that is, in part, dry, in part oily. This regimen calls for the sequential application to the skin of the face, in the morning, and in the evening, of up to five products: a daily cleanser; an exfoliating masque with thermal infusion; a daily balancer; a daily eye cream; and a daily moisturizing cream for use in the morning and in the evening.

The regimen provides a series of skin treatment steps beginning with the step of washing one's face with the daily cleanser. Subsequently, application of the exfoliating masque may be used, which is followed with the daily balancer. Thereafter, the eye cream is applied, followed by application of the moisturizing cream.

### DESCRIPTION OF THE PREFERRED EMBODIMENTS

The morning and evening skin treatment regimens utilize effective amounts of up to five defined products. Preferably, these five products are used in a series of steps, including a multi-step morning regimen using at least four of the five products, and a multi-step evening regimen using at least four of the five products.

Because these products use some common ingredients, the following summary pinpoints certain select ingredients which uniquely enhance the effectiveness of the present skin care regimen. The names used for the ingredients herein include proprietary names, the contents of which are defined and terms derived from the International Nomenclature for Cosmetic Ingredients (INCI), as well as commonly used terms.

The following lists summarize the key ingredients of the five skin care products of the present invention, and offer a complete list of the ingredients to set forth the best mode of the invention, including recommended weight percentages. Additionally, some ingredients listed separately below are noted to be part of a blend. In such cases, the listed percent represents the percent of that blend within a particular product. Therefore, the actual percentages of products comprising that blend is lower than the listed percentage. Small differences in percentages are equivalent amounts envisioned within the spirit and scope of the claimed invention. In particular, the weight percentages of the key ingredients may vary about plus or minus 30%, depending on the user's objectives, age and skin condition. Thus, the invention should not be limited by each specific list of ingredients nor the specified concentrations.

Product 1: Daily Cleanser:

The key ingredients of the daily cleanser include: bio-hydria complex, including comfrey, ginseng, cucumber, birch leaf, watercress, clover blossom, St. John's wort extracts (together, these ingredients soften, cool, soothe, condition, and normalize skin); oat amino acids (mild cleanser); and decyl glucoside (mild cleanser; breaks down oil and dirt).

The following is an example of the daily cleanser:

(Quantitative Formula)

| Ingredients | % |
|---|---|
| Water | >60 |
| Magnesium Aluminum Silicate | 0.1-0.9 |
| Disodium EDTA | 0.01-0.1 |
| Sodium Cocoyl Isethionate | 15-30 |
| Stearic Acid | |
| Glycol Stearate | 1-3 |
| Decyl Glucoside | 1-3 |
| Oleamidopropyl Betaine | 3-5 |
| Lauramide DEA | 2-4 |
| Lecithin | 0.1-0.5 |
| Citric Acid | 0.1-0.5 |
| Sodium Lauroyl Oat Amino Acids | 0.1-0.5 |
| Symphytum Officinale Extract | 0.001-0.005 |
| Cucumis Sativus (Cucumber) Fruit Extract | 0.001-0.005 |
| Nasturtium Officinale Extract | 0.001-0.005 |
| Betula Alba Leaf Extract | 0.001-0.005 |
| Trifolium Pratense (Clover) Flower Extract | 0.001-0.005 |
| Panax Ginseng Root Extract | 0.001-0.005 |
| Hypericum Perforatum Extract | 0.001-0.005 |
| Sodium PCA | 0.001-0.005 |
| Polyamino Sugar Condensate | 0.001-0.005 |
| Urea | |
| Panthenol | 0.1-0.5 |
| Hydrolyzed Soy Protein | 0.1-0.5 |
| Potassium Sorbate | 0.1-0.5 |
| Menthol | 0.1-0.5 |
| Methylchloroisothiazolinone | 0.01-0.05 |
| Phospholipids | 0.5-1 |
| Tocopheryl Acetate | |
| Retinyl Palmitate | |
| Ascorbyl Palmitate | |
| Sodium Chloride | 0.1-0.5 |
| Citrus Medica Limonum (Lemon) Fruit Extract | 0.1-0.2 |
| Ricinus Communis (Castor) Seed Oil | |
| Citrus Aurantium Dulcis (Orange) Oil | |
| Geraniol | |
| Citrus Aurantium Bergamia (Bergamot) Fruit Oil | |
| Salvia Sclarea (Clary) Oil | |
| Litsea Cubeba Fruit Oil | |
| Elettaria Cardamomum Seed Oil | |

Product 2: Exfoliating Masque with Thermal Infusion:

The key ingredients of the exfoliating masque with thermal infusion are: bio-hydria complex, including comfrey, ginseng, cucumber, birch leaf, watercress, clover blossom, St. John's wort extracts (together, these ingredients soften, cool, soothe, condition, and normalize skin); papain (unripened papaya enzyme used to gently exfoliate skin); walnut shell powder (sloughs off dead skin cells); benzyl nicotinate (stimulates circulation and enzyme activity (from papain); kaolin/bentonite (draws out impurities to bring them closer to the surface for easy removal); liposomes (small enough to penetrate the epidermis (200 nanometers; 1 nanometer equals 1 billionth of a meter, infused with beneficial ingredients and are ready to transport them to the epidermis or dermis); and decyl glucoside (mild cleanser; breaks down oil and dirt).

The following is an example of the exfoliating masque with thermal infusion:

(Quantitative Formula)

| Ingredients | % |
|---|---|
| Water | >50 |
| Bentonite | 8-10 |
| Kaolin | 20-25 |
| Glycerin | 5-6 |
| Disodium EDTA | 0.01-0.05 |
| Titanium Dioxide | 0.5-1 |
| Juglans Regia (Walnut) Shell Powder | 3-5 |
| Hydrolyzed Soy Protein | 0.1-0.5 |
| Symphytum Officinale Extract | 0.005-0.1 |
| Cucumis Sativus (Cucumber) Fruit Extract | 0.005-0.1 |
| Nasturtium Officinale Extract | 0.005-0.1 |
| Betula Alba Leaf Extract | 0.005-0.1 |
| Trifolium Pratense (Clover)Flower Extract | 0.005-0.1 |
| Panax Ginseng Root Extract | 0.005-0.1 |
| Hypericum Perforatum Extract | 0.005-0.1 |
| Sodium PCA | 0.3-0.5 |
| Polyamino Sugar Condensate | 0.3-0.5 |
| Urea | |
| Phenoxyethanol | 0.5-1 |
| Methylchloroisothiazolinone | 0.05-0.1 |
| Decyl Glucoside | 3-5 |
| Benzyl Nicotinate | 0.03-0.05 |
| Phospholipids | 0.1-0.5 |
| Tocopheryl Acetate | |
| Retinyl Palmitate | |
| Ascorbyl Palmitate | |
| Papain | 0.01-0.05 |
| Citrus Medica Limonum (Lemon) Fruit Extract | 0.1-0.2 |
| Ricinus Communis (Castor) Seed Oil | |
| Citrus Aurantium Dulcis (Orange) Oil | |
| Geraniol | |
| Citrus Aurantium Bergamia (Bergamot) Fruit Oil | |
| Salvia Sclarea (Clary) Oil | |
| Litsea Cubeba Fruit Oil | |
| Elettaria Cardamomum Seed Oil | |

Product 3: Daily Balancer:

The key ingredients of the daily balancer are: bio-hydria complex, including comfrey, ginseng, cucumber, birch leaf, watercress, clover blossom, St. John's wort extracts (together, these ingredients soften, cool, soothe, condition, and normalize skin); blue copper (enhances skin clarity, increases the skin's moisture—without the shine—resulting in smoother skin; protects against free radicals); zinc (promotes collagen synthesis in the dermis; lowers sebaceous secretion); alpha hydroxy acids (fruit acids used to exfoliate and moisturize skin); and phospholipids (very compatible with skin lipids; great moisturizers).

The following is an example of the daily balancer:

(Quantitative Formula)

| Ingredients | % |
|---|---|
| Water | >90 |
| Disodium EDTA-Copper | 0.05-0.1 |
| Zincidone | 0.1-0.5 |
| Sodium PCA | 1-3 |
| Symphytum Officinale Extract | 0.005-0.05 |
| Cucumis Sativus (Cucumber) Fruit Extract | 0.005-0.05 |
| Nasturtium Officinale Extract | 0.005-0.05 |
| Betula Alba Leaf Extract | 0.005-0.05 |
| Trifolium Pratense (Clover) Flower Extract | 0.005-0.05 |
| Panax Ginseng Root Extract | 0.005-0.05 |
| Hypericum Perforatum Extract | 0.005-0.05 |
| Polyamino Sugar Condensate | 0.5-1 |
| Urea | |
| Butylene Glycol | |
| Lactic Acid | 0.2-0.5 |
| Potassium Sorbate | 0.2-0.5 |
| Hamamelis Virginiana (Witch Hazel) Extract | 0.1-0.5 |
| Phenoxyethanol | 0.5-1 |
| Polysorbate 20 | 2-5 |
| Ascorbyl Palmitate | 0.5-1 |
| Tocopheryl Acetate | 0.005-0.5 |
| Retinyl Palmitate | 0.005-0.5 |
| Citrus Medica Limonum (Lemon) Fruit Extract | 0.1-0.2 |
| Ricinus Communis (Castor) Seed Oil | |
| Citrus Aurantium Dulcis (Orange) Oil | |
| Geraniol | |
| Citrus Aurantium Bergamia (Bergamot) Fruit Oil | |
| Salvia Sclarea (Clary) Oil | |
| Litsea Cubeba Fruit Oil | |
| Elettaria Cardamomum Seed Oil | |
| Cocamidopropyl PG-Dimonium Chloride Phosphate | 1-3 |

Product 4: Daily Eve Cream:

The key ingredients of the daily eye cream are: bio-hydria complex, including comfrey, ginseng, cucumber, birch leaf, watercress, clover blossom, St. John's wort extracts (together, these ingredients soften, cool, soothe, condition, and normalize skin); alpha hydroxy acids (fruit acids used to exfoliate and moisturize skin); lysate extract (skin calmer and soothing agent); light diffuser (reflects light to distract from any skin imperfections); and liposomes (small enough to penetrate the epidermis (200 nanometers; 1 nanometer equals 1 billionth of a meter; infused with beneficial ingredients and are ready to transport them to the epidermis or dermis).

The following is an example of the daily eye cream:

(Quantitative Formula)

| Ingredients | % |
|---|---|
| Water | >70 |
| Disodium EDTA | 0.05-0.1 |
| Glycerin | 3-5 |
| Cellulose Gum | 0.1-0.5 |
| Phenoxyethanol | 0.5-1 |
| Potassium Sorbate USP | 0.1-0.3 |
| Lactic Acid | 0.1-0.3 |
| Caprylic/Capric Triglyceride | 1-3 |
| Stearic Acid | 0.5-1 |
| Cetyl Alcohol | 0.5-1 |
| Steareth-21 | 1-2 |
| Steareth-2 | 1-2 |
| Cetyl Lactate | 1-2 |
| Tocopheryl Nicotinate | 0.2-0.4 |
| Lecithin | 0.1-0.4 |
| Ethylene/Acrylic Acid Copolymer | 1-3 |
| Symphytum Officinale Extract | 0.005-0.1 |
| Cucumis Sativus (Cucumber) Fruit Extract | 0.005-0.1 |
| Nasturtium Officinale Extract | 0.005-0.1 |
| Betula Alba Leaf Extract | 0.005-0.1 |
| Trifolium Pratense (Clover) Flower Extract | 0.005-0.1 |
| Panax Ginseng Root Extract | 0.005-0.1 |
| Hypericum Perforatum Extract | 0.005-0.1 |
| Sodium PCA | 0.3-0.5 |
| Polyamino Sugar Condensate | 0.3-0.5 |
| Urea | |
| Butylene Glycol | |
| Hydrolyzed Soy Protein | 0.1-0.3 |
| Phospholipids | 0.05-0.2 |
| Tocopheryl Acetate | |
| Retinyl Palmitate | |
| Ascorbyl Palmitate | |
| Saccharomyces Lysate Extract | 1-2 |
| Polysorbate 20 | 1-2 |
| Retinyl Acetate | |
| Methylchlorisothiazolinone | 0.01-0.05 |
| Polyacrylate 13 | 1-3 |
| Polyisobute | |

Product 5: Daily Moisturizing Cream, Day & Night:

The key ingredients of the daily moisturizing cream, day & night are: bio-hydria complex, including comfrey, ginseng, cucumber, birch leaf, watercress, clover blossom, St. John's wort extracts (together, these ingredients soften, cool, soothe, condition, and normalize skin; alpha hydroxy acids (fruit acids used to exfoliate and moisturize skin); and liposomes (small enough to penetrate the epidermis (200 nanometers; 1 nanometer equals 1 billionth of a meter; infused with beneficial ingredients, and capable of transporting them to the epidermis or dermis).

The following is an example of the daily moisturizing cream, day & night:

(Quantitative Formula)

| Ingredients | % |
|---|---|
| Water | >70 |
| Glycerin | 3-5 |
| Cellulose Gum | 0.3-0.5 |
| Phenoxyethanol | 0.5-1 |
| Potassium Sorbate | 0.05-0.1 |
| Lactic Acid | 0.2-0.4 |
| Disodium EDTA | 0.05-0.1 |
| C12-15 Benzyl Benzoate | 1-3 |
| Caprylic/Capric Triglyceride | 1-3 |
| Stearic Acid | 1-3 |
| Cetyl Alcohol | 1-3 |
| Steareth-21 | 1-3 |
| Steareth-2 | 1-3 |
| Cetyl Lactate | 1-3 |
| Lecithin | 0.1-0.5 |
| Symphytum Officinale Extract | 0.005-0.05 |
| Cucumis Sativus (Cucumber) Fruit Extract | 0.005-0.05 |
| Nasturtium Officinale Extract | 0.005-0.05 |
| Birch Betula Alba Leaf Extract | 0.005-0.05 |
| Trifolium Pratense (Clover) Flower Extract | 0.005-0.05 |
| Panax Ginseng Root Extract | 0.005-0.05 |
| Hypericum Perforatum Extract | 0.005-0.05 |
| Sodium PCA | 0.5-1 |
| Polyamino Sugar Condensate | 0.5-1 |
| Urea | |
| Butylene Glycol | |
| Hydrolyzed Soy Protein | 0.1-0.5 |
| Methylchloroisothiazolinone | 0.050-0.1 |
| Phospholipids | 1-2 |
| Tocopheryl Acetate | |
| Retinyl Palmitate | |
| Ascorbyl Palmitate | |
| Polysorbate 20 | |
| Polyacrylate 13 | 1-2 |
| Polyisobutene | |
| Citrus Medica Limonum (Lemon) Fruit Extract | 0.1-0.2 |
| Ricinus Communis (Castor) Seed Oil | |
| Citrus Aurantium Dulcis (Orange) Oil | |
| Geraniol | |
| Citrus Aurantium Bergamia (Bergamot) Fruit Oil | |
| Salvia Sclarea (Clary) Oil | |
| Litsea Cubeba Fruit Oil | |
| Elettaria Cardamomum Seed Oil | |

Preferably, a user applies the products in a daily regimen to enhance skin appearance, including a five-step morning regimen and the same five-step evening regimen.

Step 1. Apply the daily cleanser (Product 1) by applying a small amount of the cleanser on moist skin and massaging gently in circular motions. Excess daily cleanser is rinsed off after application.

Step 2. After application of the daily cleanser, apply the exfoliating masque with thermal fusion (Product 2) directly to the face.

Step 3. After application of the exfoliating masque with thermal fusion, apply the daily balancer (Product 3) liberally onto skin and massage in a circular motion. The daily balancer is not rinsed off or wiped off after application, but remains on the face.

Step 4. After application of the daily balancer, apply daily eye cream (Product 4) using the ring finger. Dot around eyes and gently pat until product is absorbed. The daily eye cream is not rinsed off after application.

Step 5: After application of the daily eye cream, apply daily moisturizing cream, day & night (Product 5), massaging liberally into the skin using circular motions. The daily moisturizing cream, day & night is not rinsed off after application, but remains on the face.

Within the foregoing description, many specific details commonly understood by those skilled in the art have not been recited so as to not needlessly obscure many of the essential features of the present invention. In other instances, some non-essential details of the present invention have been recited in the detailed description to better enable the reader to make and use the claimed invention. The many details within the foregoing description are therefore not intended to limit the scope of the claims appended hereto, said claims being intended to cover alternative processes, modifications, and equivalents which may be included within the spirit and scope of the foregoing description and the appended claims.

What is claimed is:

1. A skin care regimen comprising a daily cleanser, a daily balancer, a daily eye cream, and a daily moisturizer, each applied to facial skin at least once per day.

2. The skin care regimen according to claim 1 further comprising an exfoliating masque with thermal infusion, applied after said daily cleanser and before said daily balancer.

3. The skin care regimen wherein said daily cleanser comprises components that soften, condition, and normalize said facial skin.

4. The skin care regimen according to claim 3 wherein said daily cleanser comprises: bio-hydria complex, including comfrey, ginseng, cucumber, birch leaf, watercress, clover blossom, St. John's wort extracts, oat amino acids, and decyl glucoside.

5. The skin care regimen according to claim 1 wherein said daily balancer comprises components that soothe and condition said skin, enhance the clarity of said skin, promote collagen synthesis in the dermis of said skin, and exfoliate and moisturize said skin.

6. The skin care regimen according to claim 5 wherein said daily balancer comprises: bio-hydria complex, including comfrey, ginseng, cucumber, birch leaf, watercress, clover blossom, St. John's wort extracts, blue copper, zinc, alpha hydroxy acids, and phospholipids.

7. The skin care regimen according to claim 1 wherein said eye cream comprises components that soften, condition normalize, calm, soothe, exfoliate and moisturize skin, and reflect light.

8. The skin care regimen according to claim 7 wherein said eye cream comprises: bio-hydria complex, including comfrey, ginseng, cucumber, birch leaf, watercress, clover blossom, St. John's wort extracts, alpha hydroxy acids, lysate extract, light diffuser and liposomes.

9. The skin care regimen according to claim 1 wherein said daily moisturizer comprises ingredients that soften, condition, exfoliate and moisturize said facial skin.

10. The skin care regimen according to claim 9, wherein said daily moisturizer comprises: bio-hydria complex, including comfrey, ginseng, cucumber, birch leaf, watercress, clover blossom, St. John's wort extracts, and liposomes.

11. The skin care regimen according to claim 2, wherein said exfoliating masque includes ingredients that soften, condition, cleanse, and exfoliate skin, and that stimulate circulation and enzyme activity in said facial skin.

12. The skin care regimen according to claim 11 wherein said exfoliating masque comprises: bio-hydria complex, including comfrey, ginseng, cucumber, birch leaf, watercress, clover blossom, St. John's wort extracts, papain, walnut shell powder, benzyl nicotinate, kaolin/bentonite, liposomes, and decyl glucoside.

13. The skin care regimen according to claim 1 wherein said daily cleanser comprises the following:

| Ingredients | Weight % |
|---|---|
| Water | >60 |
| Magnesium Aluminum Silicate | 0.1-0.9 |
| Disodium EDTA | 0.01-0.1 |
| Sodium Cocoyl Isethionate | 15-30 |
| Stearic Acid | |
| Glycol Stearate | 1-3 |
| Decyl Glucoside | 1-3 |
| Oleamidopropyl Betaine | 3-5 |
| Lauramide DEA | 2-4 |
| Lecithin | 0.1-0.5 |
| Citric Acid | 0.1-0.5 |
| Sodium Lauroyl Oat Amino Acids | 0.1-0.5 |
| Symphytum Officinale Extract | 0.001-0.005 |
| Cucumis Sativus (Cucumber) Fruit Extract | 0.001-0.005 |
| Nasturtium Officinale Extract | 0.001-0.005 |
| Betula Alba Leaf Extract | 0.001-0.005 |
| Trifolium Pratense (Clover) Flower Extract | 0.001-0.005 |
| Panax Ginseng Root Extract | 0.001-0.005 |
| Hypericum Perforatum Extract | 0.001-0.005 |
| Sodium PCA | 0.001-0.005 |
| Polyamino Sugar Condensate | 0.001-0.005 |
| Urea | |
| Panthenol | 0.1-0.5 |
| Hydrolyzed Soy Protein | 0.1-0.5 |
| Potassium Sorbate | 0.1-0.5 |
| Menthol | 0.1-0.5 |
| Methylchloroisothiazolinone | 0.01-0.05 |
| Phospholipids | 0.5-1 |
| Tocopheryl Acetate | |
| Retinyl Palmitate | |
| Ascorbyl Palmitate | |
| Sodium Chloride | 0.1-0.5 |
| Citrus Medica Limonum (Lemon) Fruit Extract | 0.1-0.2 |
| Ricinus Communis (Castor) Seed Oil | |
| Citrus Aurantium Dulcis (Orange) Oil | |
| Geraniol | |
| Citrus Aurantium Bergamia (Bergamot) Fruit Oil | |
| Salvia Sclarea (Clary) Oil | |
| Litsea Cubeba Fruit Oil | |
| Elettaria Cardamomum Seed Oil | |

14. The skin care regimen according to claim 2 wherein said exfoliating masque comprises the following:

| Ingredients | Weight % |
|---|---|
| Water | >50 |
| Bentonite | 8-10 |
| Kaolin | 20-25 |
| Glycerin | 5-6 |
| Disodium EDTA | 0.01-0.05 |
| Titanium Dioxide | 0.5-1 |
| Juglans Regia (Walnut) Shell Powder | 3-5 |
| Hydrolyzed Soy Protein | 0.1-0.5 |
| Symphytum Officinale Extract | 0.005-0.1 |
| Cucumis Sativus (Cucumber) Fruit Extract | 0.005-0.1 |
| Nasturtium Officinale Extract | 0.005-0.1 |
| Betula Alba Leaf Extract | 0.005-0.1 |
| Trifolium Pratense (Clover)Flower Extract | 0.005-0.1 |
| Panax Ginseng Root Extract | 0.005-0.1 |
| Hypericum Perforatum Extract | 0.005-0.1 |
| Sodium PCA | 0.3-0.5 |
| Polyamino Sugar Condensate | 0.3-0.5 |
| Urea | |
| Phenoxyethanol | 0.5-1 |
| Methylchloroisothiazolinone | 0.05-0.1 |
| Decyl Glucoside | 3-5 |
| Benzyl Nicotinate | 0.03-0.05 |
| Phospholipids | 0.1-0.5 |
| Tocopheryl Acetate | |
| Retinyl Palmitate | |
| Ascorbyl Palmitate | |
| Papain | 0.01-0.05 |
| Citrus Medica Limonum (Lemon) Fruit Extract | 0.1-0.2 |
| Ricinus Communis (Castor) Seed Oil | |
| Citrus Aurantium Dulcis (Orange) Oil | |
| Geraniol | |
| Citrus Aurantium Bergamia (Bergamot) Fruit Oil | |
| Salvia Sclarea (Clary) Oil | |
| Litsea Cubeba Fruit Oil | |
| Elettaria Cardamomum Seed Oil | |

15. The skin care regimen of claim 1 where said daily balancer comprises the following:

| Ingredients | Weight % |
|---|---|
| Water | >90 |
| Disodium EDTA-Copper | 0.05-0.1 |
| Zincidone | 0.1-0.5 |
| Sodium PCA | 1-3 |
| Symphytum Officinale Extract | 0.005-0.05 |
| Cucumis Sativus (Cucumber) Fruit Extract | 0.005-0.05 |
| Nasturtium Officinale Extract | 0.005-0.05 |
| Betula Alba Leaf Extract | 0.005-0.05 |
| Trifolium Pratense (Clover) Flower Extract | 0.005-0.05 |
| Panax Ginseng Root Extract | 0.005-0.05 |
| Hypericum Perforatum Extract | 0.005-0.05 |
| Polyamino Sugar Condensate | 0.5-1 |
| Urea | |
| Butylene Glycol | |
| Lactic Acid | 0.2-0.5 |
| Potassium Sorbate | 0.2-0.5 |
| Hamamelis Virginiana (Witch Hazel) Extract | 0.1-0.5 |
| Phenoxyethanol | 0.5-1 |
| Polysorbate 20 | 2-5 |
| Ascorbyl Palmitate | 0.5-1 |
| Tocopheryl Acetate | 0.005-0.5 |
| Retinyl Palmitate | 0.005-0.5 |
| Citrus Medica Limonum (Lemon) Fruit Extract | 0.1-0.2 |
| Ricinus Communis (Castor) Seed Oil | |
| Citrus Aurantium Dulcis (Orange) Oil | |
| Geraniol | |
| Citrus Aurantium Bergamia (Bergamot) Fruit Oil | |
| Salvia Sclarea (Clary) Oil | |
| Litsea Cubeba Fruit Oil | |
| Elettaria Cardamomum Seed Oil | |
| Cocamidopropyl PG-Dimonium Chloride Phosphate | 1-3 |

16. The skin care regimen according to claim 1 wherein said daily eye cream comprises the following:

| Ingredients | Weight % |
|---|---|
| Water | >70 |
| Disodium EDTA | 0.05-0.1 |
| Glycerin | 3-5 |
| Cellulose Gum | 0.1-0.5 |
| Phenoxyethanol | 0.5-1 |
| Potassium Sorbate USP | 0.1-0.3 |
| Lactic Acid | 0.1-0.3 |
| Caprylic/Capric Triglyceride | 1-3 |
| Stearic Acid | 0.5-1 |
| Cetyl Alcohol | 0.5-1 |
| Steareth-21 | 1-2 |
| Steareth-2 | 1-2 |
| Cetyl Lactate | 1-2 |
| Tocopheryl Nicotinate | 0.2-0.4 |
| Lecithin | 0.1-0.4 |
| Ethylene/Acrylic Acid Copolymer | 1-3 |
| Symphytum Officinale Extract | 0.005-0.1 |
| Cucumis Sativus (Cucumber) Fruit Extract | 0.005-0.1 |
| Nasturtium Officinale Extract | 0.005-0.1 |
| Betula Alba Leaf Extract | 0.005-0.1 |
| Trifolium Pratense (Clover) Flower Extract | 0.005-0.1 |
| Panax Ginseng Root Extract | 0.005-0.1 |
| Hypericum Perforatum Extract | 0.005-0.1 |
| Sodium PCA | 0.3-0.5 |
| Polyamino Sugar Condensate | 0.3-0.5 |
| Urea | |
| Butylene Glycol | |
| Hydrolyzed Soy Protein | 0.1-0.3 |
| Phospholipids | 0.05-0.2 |
| Tocopheryl Acetate | |
| Retinyl Palmitate | |
| Ascorbyl Palmitate | |
| Saccharomyces Lysate Extract | 1-2 |
| Polysorbate 20 | 1-2 |
| Retinyl Acetate | |
| Methylchlorisothiazolinone | 0.01-0.05 |
| Polyacrylate 13 | 1-3 |
| Polyisobute | |

17. The skin care regimen according to claim 1 where said daily moisturizing cream comprises the following:

| Ingredients | Weight % |
|---|---|
| Water | >70 |
| Glycerin | 3-5 |
| Cellulose Gum | 0.3-0.5 |
| Phenoxyethanol | 0.5-1 |
| Potassium Sorbate | 0.05-0.1 |
| Lactic Acid | 0.2-0.4 |
| Disodium EDTA | 0.05-0.1 |
| C12-15 Benzyl Benzoate | 1-3 |
| Caprylic/Capric Triglyceride | 1-3 |
| Stearic Acid | 1-3 |
| Cetyl Alcohol | 1-3 |
| Steareth-21 | 1-3 |
| Steareth-2 | 1-3 |
| Cetyl Lactate | 1-3 |
| Lecithin | 0.1-0.5 |
| Symphytum Officinale Extract | 0.005-0.05 |
| Cucumis Sativus (Cucumber) Fruit Extract | 0.005-0.05 |
| Nasturtium Officinale Extract | 0.005-0.05 |
| Birch Betula Alba Leaf Extract | 0.005-0.05 |
| Trifolium Pratense (Clover) Flower Extract | 0.005-0.05 |
| Panax Ginseng Root Extract | 0.005-0.05 |
| Hypericum Perforatum Extract | 0.005-0.05 |
| Sodium PCA | 0.5-1 |
| Polyamino Sugar Condensate | 0.5-1 |
| Urea | |
| Butylene Glycol | |
| Hydrolyzed Soy Protein | 0.1-0.5 |
| Methylchloroisothiazolinone | 0.050-0.1 |
| Phospholipids | 1-2 |
| Tocopheryl Acetate | |
| Retinyl Palmitate | |
| Ascorbyl Palmitate | |
| Polysorbate 20 | |
| Polyacrylate 13 | 1-2 |
| Polyisobutene | |
| Citrus Medica Limonum (Lemon) Fruit Extract | 0.1-0.2 |
| Ricinus Communis (Castor) Seed Oil | |
| Citrus Aurantium Dulcis (Orange) Oil | |
| Geraniol | |
| Citrus Aurantium Bergamia (Bergamot) Fruit Oil | |
| Salvia Sclarea (Clary) Oil | |
| Litsea Cubeba Fruit Oil | |
| Elettaria Cardamomum Seed Oil | |

18. A method of treating facial skin comprising applying to said skin, in order, at least once each day, effective amounts of a daily cleanser, a daily balancer, a daily eye cream and a daily moisturizing cream.

19. The method of claim 17 further comprising applying to said facial skin an effective amount of an exfoliating masque with thermal infusion after applying said daily cleanser to said facial skin, and before applying said daily balancer.
